(19) United States
(12) Patent Application Publication
(10) Pub. No.: US 2024/0058168 A1
(43) Pub. Date: Feb. 22, 2024

(54) CAMERA-BASED DROPLET GUIDANCE AND DETECTION

(71) Applicant: TWENTY TWENTY THERAPEUTICS LLC, South San Francisco, CA (US)

(72) Inventors: Oleg Rumyantsev, Menlo, CA (US); Supriyo Sinha, Menlo Park, CA (US); Dimitri Azar, Chicago, CA (US); Kevin Luo, South San Francisco, CA (US); Michael Eyal, South San Francisco, CA (US)

(21) Appl. No.: 18/236,375

(22) Filed: Aug. 21, 2023

Related U.S. Application Data
(60) Provisional application No. 63/399,353, filed on Aug. 19, 2022.

(51) Int. Cl. A61F 9/00; A61B 34/00

(52) U.S. Cl. CPC A61F 9/0026; A61B 34/76; A61B 2034/2057

(57) ABSTRACT

Systems and methods are provided for determining a likelihood that a drop released from a bottle will land in an eye of a user. Each of an eye of a user and a tip of a bottle and illuminated and imaged to provide at least one image. An orientation of the bottle is determined via an inertial measurement unit, and a likelihood that a drop released from the bottle will land in the eye of the user is determined from the determined orientation and the at least one image.

### CROSS REFERENCE TO RELATED APPLICATION

This application claims priority from each of U.S. Provisional Patent Application Ser. No. 63/399,353, filed Aug. 19, 2022, and entitled “Camera-Based Droplet Guidance and Detection.” This application is hereby incorporated by reference in its entirety.

### TECHNICAL FIELD

The disclosure relates generally to the field of medical systems, and more particularly to camera-based droplet guidance and detection.

### BACKGROUND

Patients, especially elderly patients, have difficulty applying prescribed medication to their eyes, in particular confirming that the application of drops was successful and following the application protocol (e.g., time of day, number of drops, etc.). This leads to worsened treatment outcomes and discourages doctors from prescribing optimal treatment protocols. Further, optimal positioning of an eye drop bottle can be difficult for patients; leading to wasted medication or incomplete application of the medication to the eye.

### SUMMARY

In accordance with one example, a method is provided. Each of an eye of a user and a tip of a bottle are illuminated and imaged to provide at least one image. A relative location of the eye of the user and the tip of the bottle is determined from the at least one image. An orientation of the bottle is determined via an inertial measurement unit, and a likelihood that a drop released from the bottle will land in the eye of the user is determined from the determined orientation and the at least one image.

In accordance with another example, a system includes an illumination source positioned to illuminate each of an eye of a user and a tip of a bottle when the when the device and the bottle with which it is associated is in an appropriate position to deliver mediation to the eye. A camera images each of the eye of the user and the tip of the bottle while the illumination source is active to provide at least one image. A release of a drop from the bottle is detected from the image or images. An inertial measurement unit that determines an orientation of the bottle. An alignment component determines a likelihood that a drop released from the bottle will land in the eye of the user from the determined orientation of the bottle and the at least one image.

In accordance with a further example, a method is provided for monitoring compliance in the application of eye drops for a user. Each of an eye of a user and a tip of a bottle are illuminated and imaged to provide at least one image. A relative location of the eye of the user and the tip of the bottle is determined from the at least one image. An orientation of the bottle is determined via an inertial measurement unit, and a likelihood that a drop released from the bottle will land in the eye of the user is determined from the determined orientation and the at least one image in response to detecting the release of the drop from the bottle.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a device for guiding the application of eye drops for a user;

FIG. 2 illustrates one example of a device for monitoring compliance in the application of eye drops for a user;

FIG. 3 is a schematic hardware diagram of one implementation of a device as described in FIGS. 1 and 2;

FIG. 4 illustrates a method for determining if a released from a bottle with an eye dropper is likely to land in an eye of a user;

FIG. 5 illustrates a method for monitoring compliance of user with a treatment protocol involving administration of drops to the eye of the user;

FIG. 6 illustrates a method for determining if a released from a bottle with an eye dropper is likely to land in an eye of a user; and

FIG. 7 is a schematic block diagram illustrating an example system of hardware components capable of implementing examples of the systems and methods disclosed herein.

### DETAILED DESCRIPTION

As used herein, subtracting a first image from a second image refers to a pixel-by-pixel subtraction of one or more chromaticity values associated with each pixel in the first image from the associated value or values of a corresponding pixel in the second image.

As used herein, a “droplet” is a small drop of fluid, and the terms “drop” and “droplet” are used interchangeably to describe such a drop of fluid.

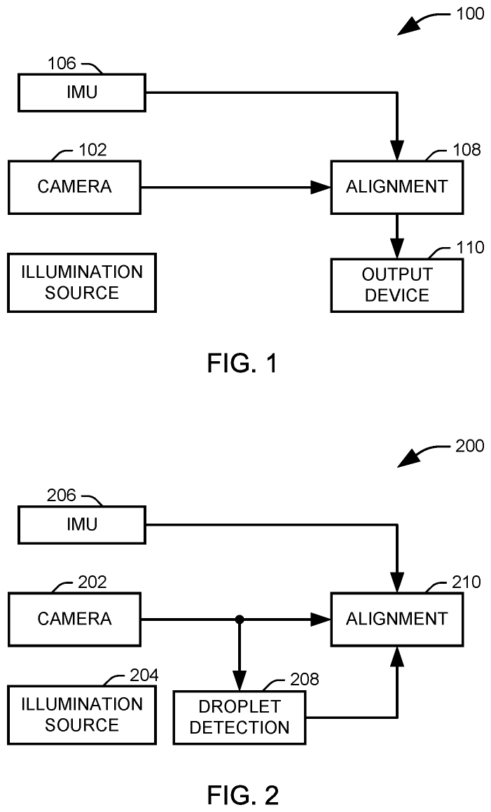

Various examples of the systems and methods described herein allow for guiding a user in applying eye drops and verifying their successful application. FIG. 1 illustrates a device 100 for guiding the application of eye drops for a user. It will be appreciated that the device 100 can be integral with a bottle containing medication intended for application to the user's eye or implemented as a stand-alone device that can be mounted onto a bottle having known dimensions and a known configuration. In one implementation, the device 100 is configured to be attached to a standard prescription eye dropper bottle. The device 100 includes a camera 102 that is positioned to capture a tip of a bottle (not shown) associated with the eye drop bottle, as well as an eye of the user within a field of view of the camera when the device and the bottle with which it is associated is in an appropriate position to deliver mediation to the eye.

In the illustrated implementation, the camera 102 can include a spectral filter that attenuates light outside of a narrow band of wavelengths. In this implementation, an illumination source 104 can be positioned to illuminate each of the tip of the bottle and the eye with light having a wavelength within the narrow band of wavelengths. It will be appreciated that the illumination source 104 can include multiple individual light sources that are controlled independently to illuminate the tip of the bottle and the eye at different times and intensities. In one example, the illumination source provides infrared light of a specific wavelength, and the spectral filter on the camera 102 is selected to attenuate light within the visible spectrum. In another example, no spectral filter is used with the camera 102, but the illumination source 104 is modulated to pulse in synchrony with a frame acquisition rate of the camera 102, with unilluminated frames subtracted from illuminated frames to remove the contribution of other portions of the spectrum to as part of a background subtraction process. For example, for a camera operating at forty frames per second, the illumination source 104 can be pulsed at a rate of twenty hertz with a pulse length of twenty-five milliseconds, and every other frame can be subtracted from an adjacent, illuminated frame to provide the background subtraction.

An inertial measurement unit (IMU) 106 tracks an orientation of the device 100 in space relative to a reference direction, for example, the direction of gravitational force. An alignment component 108 processes images received from the camera 102 to determine an alignment of the tip of the bottle with the eye, in particular, if a drop released from the tip of the bottle is likely to land in the eye. In one implementation, the alignment component 108 is implemented as dedicated hardware components, for example, implemented as an application-specific integrated circuit, that utilize an image or images from the camera 102 and the output of the IMU 106 to determine the alignment. In another example, the alignment component 108 is implemented as a processor and a non-transitory computer readable medium that stores executable instructions for determining the alignment from the output of the IMU 106 and the camera 102. In the illustrated implementation, the alignment component 108 is implemented on the device 100, but in some implementations, the alignment component 108 can be implemented on another device, for example, a mobile device carried by the user, and the data from the camera 102 and the IMU 106 can be transmitted to the alignment component 108 via a short-range wireless communication protocol, such as Bluetooth.

In one implementation, the alignment component 108 includes an image processing algorithm that determines a likelihood that a drop released from the bottle will land in the eye of the patient given the outputs of the IMU 106 and the camera 102. For example, the image processing algorithm can apply a number of image processing techniques to one or more images to identify a reflection of the illumination source 104 in the cornea of the eye, including a normalization process, morphological processing, filtering, and clustering. Alternatively or additionally, the alignment component 108 can apply a landmark fitting algorithm to locate the boundary of the eyelid and the iris, with the size and shape of the iris used to establish a location for the eye. Once a location for the eye relative to the bottle is established, the location of the eye and the position of the bottle relative to gravity can be used to determine a likelihood that the drop will land within the eye if a drop is released while the bottle is in its current position. This can take into account both the expected direction for the drop to fall, given the orientation detected at the IMU 106 as well as the stability of the bottle, based on a window of data collected at the IMU. This can detect tremors or other unsteadiness in the hands of the individual delivering the drops that might negatively impact the likelihood that the drop will land within eye. This can be represented, for example, as a decreased confidence in the trajectory of the drop, such that alignment of the bottle with the eye near a boundary of the exposed area of the eye can be penalized.

In an alternative implementation, the output of the IMU 106 and one or more images from the camera 102 are provided to a machine learning model that outputs the likelihood that the drop will land within the eye if a drop is released while the bottle is in its current position. It will be appreciated that the alignment component 108 can update the likelihood continuously as new information is received from the camera 102 and the IMU 106, to provide a time series of likelihoods that a released drop will land in the eye. The machine learning model 110 can utilize one or more pattern recognition algorithms, each of which may analyze images from the camera 102 or numerical features extracted from the images with orientation data provided via the IMU 106 to assign a continuous or categorical parameter to the likelihood that a released drop would land in the eye. Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. For rule-based models, such as decision trees, domain knowledge, for example, as provided by one or more human experts, can be used in place of or to supplement training data in selecting rules for classifying a user using the extracted features. Any of a variety of techniques can be utilized for the classification algorithm, including support vector machines (SVM), regression models, self-organized maps, fuzzy logic systems, data fusion processes, boosting and bagging methods, rule-based systems, or artificial neural networks (ANN).

For example, an SVM classifier can utilize a plurality of functions, referred to as hyperplanes, to conceptually divide boundaries in the N-dimensional feature space, where each of the N dimensions represents one associated feature of the feature vector. The boundaries may define a range of feature values associated with each class. Accordingly, a continuous or categorical output value can be determined for a given input feature vector according to its position in feature space relative to the boundaries. In one implementation, the SVM can be implemented via a kernel method using a linear or non-linear kernel. A trained SVM classifier may converge to a solution where the optimal hyperplanes have a maximized margin to the associated features.

An ANN classifier may include a plurality of nodes having a plurality of interconnections. The values from the feature vector may be provided to a plurality of input nodes. The input nodes may each provide these input values to layers of one or more intermediate nodes. A given intermediate node may receive one or more output values from previous nodes. The received values may be weighted according to a series of weights established during the training of the classifier. An intermediate node may translate its received values into a single output according to a transfer function at the node. For example, the intermediate node can sum the received values and subject the sum to a rectifier function. The output of the ANN can be a continuous or categorical output value. In one example, a final layer of nodes provides the confidence values for the output classes of the ANN, with each node having an associated value representing a confidence for one of the associated output classes of the classifier. The confidence values can be based on a loss function such as a cross-entropy loss function. The loss function can be used to optimize the ANN. In an example, the ANN can be optimized to minimize the loss function.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier may apply a set of logical rules to the extracted features to select an output class. The rules may be applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees may be trained on random samples of the training set, and an average (e.g., mean, median, or mode) result across the plurality of decision trees is returned. For a classification task, the result from each tree would be categorical, and thus a modal outcome can be used.

The alignment component 108 provides the determined likelihood to an output device 110. In one implementation, the output device 110 is implemented as one or more light sources positioned to be visible to a user, and one of an intensity, hue, or blink frequency of the light can be altered to indicate the likelihood that a released drop will land in the eye. For example, the determined likelihood can be mapped to a series of colors that represent the suitability of the alignment of the bottle with the eye, with one color (e.g., green) indicating a good alignment, a second color (e.g., red) indicating an unsuitable alignment, and a third color (e.g., yellow) representing an alignment intermediate between a good alignment and a poor alignment. It will be appreciated that gradations between these colors can be displayed to allow for a more granular display of the determined likelihood. In another example, a display can be provided to give the likelihood or a categorical parameter representing likelihood.

In a further example, the output device 110 is implemented as a speaker that provides audible feedback representing the determined likelihood, for example, by varying a volume, pitch, pulse width, or pulse rate of an audio tone, or by providing synthesized speech output. In a still further example, tactile feedback can be provided, with an intensity or frequency of the tactile feedback alerted in accordance with the determined probability. It will be appreciated that any of tactile, visual, or auditory feedback can be provided in a binary fashion with feedback provided only when the likelihood that a released drop will land in the eye. In a still further example, the output device 110 can be a mechanism for automated squeezing of the bottle to release a drop in response to a determination that the likelihood of a released drop landing in the eye exceeds a threshold value. This can be particularly helpful for patients with muscle weakness or tremors that might complicate squeezing the bottle while maintaining a suitable alignment of the bottle with the eye. Additionally or alternatively, the output device 110 can include a transceiver for communicating with another device, such as a portable computing device (e.g., tablet or mobile phone) associated with the user. This connection can be used to provide information about alignment, success or failure of the drop, and the time of application of the drop to the user's device, a caretaker's device, or a physician, for example, via an Internet connection on the portable computing device. In one example, communication is performed via low energy Bluetooth.

FIG. 2 illustrates one example of a device 200 for monitoring compliance in the application of eye drops for a user. It will be appreciated that the device 200 can be integral with a bottle containing medication intended for application to the user's eye or implemented as a stand-alone device that can be mounted onto a bottle having known dimensions and a known configuration. In one implementation, the device 200 is configured to be attached to a standard prescription eye dropper bottle. The device 200 includes a camera 202 that is positioned to capture a tip of a bottle (not shown) associated with the device, as well as an eye of the user within a field of view of the camera when the device and the bottle with which it is associated is in an appropriate position to deliver mediation to the eye.

In the illustrated implementation, the camera 202 can include a spectral filter that attenuates light outside of a narrow band of wavelengths. In this implementation, an illumination source 204 can be positioned to illuminate each of the tip of the bottle and the eye with light having a wavelength within the narrow band of wavelengths. It will be appreciated that the illumination source 204 can include multiple individual light sources that are controlled independently to illuminate the tip of the bottle and the eye at different times and intensities. In one example, the illumination source provides infrared light of a specific wavelength, and the spectral filter on the camera 202 is selected to attenuate light within the visible spectrum. In another example, the illumination source 204 is modulated to pulse in synchrony with a frame acquisition rate of the camera 202, with unilluminated frames subtracted from illuminated frames to remove the contribution of other portions of the spectrum to as part of a background subtraction process. For example, for a camera operating at forty frames per second, the illumination source 204 can be pulsed at a rate of twenty hertz with a pulse length of twenty-five milliseconds, and every other frame can be subtracted from an adjacent, illuminated frame to provide the background subtraction.

An inertial measurement unit (IMU) 206 tracks an orientation of the device 200 in space relative to a reference direction, for example, the direction of gravitational force. A droplet detection component 208 processes images received from the camera 202 to determine if a drop has been released from the tip of the bottle. In one implementation, the droplet detection component 208 segments a region of interest containing the tip of the bottle from each image provided by the camera. The region of interest is blurred, for example, via convolution with a gaussian or uniform structuring element and subtracted from the original ROI, leaving only features with high spatial frequencies. A thresholding process is applied separate the regions corresponding to the LED reflections in the droplet, and each processed ROI is then convolved with a predefined weight matrix and summed over all pixels to get a score. The score for each image is processed real-time by passing it through a peak-detection algorithm, with peaks in the score trace corresponding to the times at which a drop was released from the bottle. A time associated with each detected drop can be recorded in a non-transitory storage medium (not shown) associated with the device 200 or transmitted via a short-range wireless communication protocol, such as Bluetooth to another device.

In the illustrated implementation, the device 200 includes an alignment component 210 that determines an alignment of the tip of the bottle with the eye, in particular, if a drop released from the tip of the bottle is likely to land in the eye. In one example, the alignment component 210 is implemented as dedicated hardware components, for example, implemented as an application-specific integrated circuit, that utilize an image or images from the camera 202 associated with the time that the drop was released and the output of the IMU 206 to determine the alignment. In another example, the alignment component 210 is implemented as a processor and a non-transitory computer readable medium that stores executable instructions for determining the alignment from the output of the IMU 206 and the camera 202 at the time the drop was released. In the illustrated implementation, the alignment component 210 is implemented on the device 200, but in some implementations, the alignment component 210 can be implemented on another device, for example, a mobile device carried by the user, and the data from the camera 202 and the IMU 206 can be transmitted to the alignment component 210 via the short-range wireless communication protocol along with the time at which the drop was released.

In one implementation, the alignment component 210 includes an image processing algorithm that determines a likelihood that the drop released from the bottle will land in the eye of the patient given the outputs of the IMU 206 and the camera 202 at the time of release. For example, the image processing algorithm can apply a number of image processing techniques, similar to those applied by the drop detection component 208, to one or more images to identify a reflection of the illumination source 204 in the cornea of the eye. Alternatively or additionally, the alignment component 210 can apply a landmark fitting algorithm to locate the boundary of the eyelid and the iris, with the size and shape of the iris used to establish a location for the eye. Once a location for the eye relative to the bottle is established, the location of the eye and the position of the bottle relative to gravity can be used to determine a likelihood that the drop will land within the eye. This can take into account both the expected direction for the drop to fall, given the orientation detected at the IMU 206 as well as the stability of the bottle, based on a window of data collected at the IMU. This can detect tremors or other unsteadiness in the hands of the individual delivering the drops that might negatively impact the likelihood that the drop will land within eye. This can be represented, for example, as a decreased confidence in the trajectory of the drop, such that alignment of the bottle with the eye near a boundary of the exposed area of the eye can be penalized. In an alternative implementation, the output of the IMU 206 and one or more images from the camera 202 are provided to a machine learning model that outputs the likelihood that the released drop will land within the eye. The determined likelihood, or a categorical parameter representing the determined likelihood, can be communicated to the user, via an output device or electronic message, as well as recorded or transmitted to another device with the time at which the drop was released.

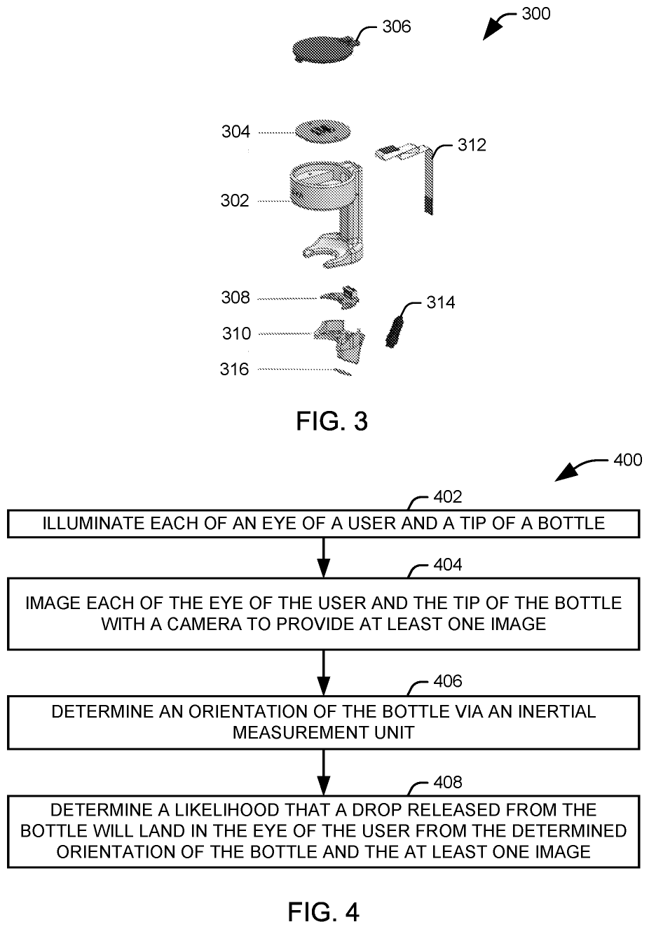

FIG. 3 is a schematic hardware diagram 300 of one implementation of a device as described in FIGS. 1 and 2. The device includes a main housing 302 configured to engage with an eye dropper bottle having known dimensions. The main housing 302 is configured to receive a first printed circuit board assembly (PCBA) 304, which is protected by a cap 306, and a second PCBA 308, which is protected by a secondary housing 310. In one implementation, either or both of the main housing 302 and the secondary housing 310 can include eye-cups or anchor points that touch the face of the user to help position the device and keep it steady during drop application. The two PCBAs 304 and 308 are connected by a flexible printed circuit 312 that runs inside the main housing 302. It will be appreciated that the first PCBA 304, the second PCBA 308, and the flexible printed circuit 312 can collectively implement all or part of any of the IMU 106 or 206, the alignment component 108 or 210, the droplet detection component 208, and any short-range communication protocol from the devices of FIGS. 1 and 2.

A camera 314 engages with the secondary housing 310, which maintains the camera in a position in which a field of view of the camera encompasses a tip of any bottle placed within the device. A spectral filter 316 can be positioned to attenuate light outside of a specified band of wavelengths, generally centered around a wavelength associated with an illumination source (not shown) of the device. In one example, the illumination source is implemented as two infrared light emitting diodes that are mounted on the underside of the secondary housing. In this implementation, the image is processed to locate the reflection of the two light emitting diodes in the eye and any released droplet. Additionally or alternatively, the illumination source is mounted to the underside of the main housing 302, directly above the expected location of a neck of the bottle, to illuminate any released drop via scattered light that passes through the bottle walls. This illumination forms a distinct arc at the bottom of the drop and does not illuminate the face of the user, improving the fidelity of drop detection, as there is no background illumination. In one example, light emitting diodes are placed in both locations, with light emitting diodes on the secondary housing 310 used for eye position estimation and light emitting diodes on the main housing used for indirect illumination for drop detection. The two sets of light emitting diodes can be pulsed asynchronously but in sync with the camera framerate. For example, for camera imaging at forty hertz, drop illumination LEDs may be turned on for odd frames, and the eye illumination LEDs for even frames. The two types of frames are then sent into different processing streams for drop detection and eye tracking.

In view of the foregoing structural and functional features described above, example methods will be better appreciated with reference to FIGS. 4-6. While, for purposes of simplicity of explanation, the example methods of FIGS. 4-6 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 4 illustrates a method 400 for determining if a released from a bottle with an eye dropper is likely to land in an eye of a user. At 402, each of an eye of a user and a tip of a bottle are illuminated. At 404, each of the eye of the user and the tip of the bottle are imaged with a camera to provide at least one image. In one example, each of eye of the user and the tip of the bottle are illuminated with light of a specific wavelength, and imaged through a spectral filter that attenuates light outside of a band of wavelengths including the specific wavelength. In another example, an illumination source is pulsed in synchrony with a frame acquisition rate of the camera to produce a plurality of illuminated images and a plurality of non-illuminated images, with an unilluminated image from each illuminated image to provide a background subtracted image during image processing. In a further example, the eye of the user is illuminated with a first illumination source and the tip of the bottle is illuminated with a second illumination source, with the first illumination source being inactive at least a portion of a time for which the second illumination source is active. For example, the tip of the bottle can be illuminated and imaged while the eye is not illuminated to reduce the effects of background illumination on images of the tip of the bottle.

At 406, an orientation of the bottle is determined via an inertial measurement unit. At 408, a likelihood that a drop released from the bottle will land in the eye of the user is determined from the determined orientation of the bottle and the at least one image. In one implementation, the at least one image and the determined orientation are provided to a machine learning model trained on previous data labeled with known outcomes to provide the likelihood that the drop will land in the eye. In another implementation, a relative location of the eye of the user and the tip of the bottle from the at least one image, and that relative location and the orientation of the bottle are used to determine the likelihood. The determined likelihood can be provided to the user via an output device, used to automatically trigger a release of a drop, or stored in a local or remote memory to represent compliance with a course of care.

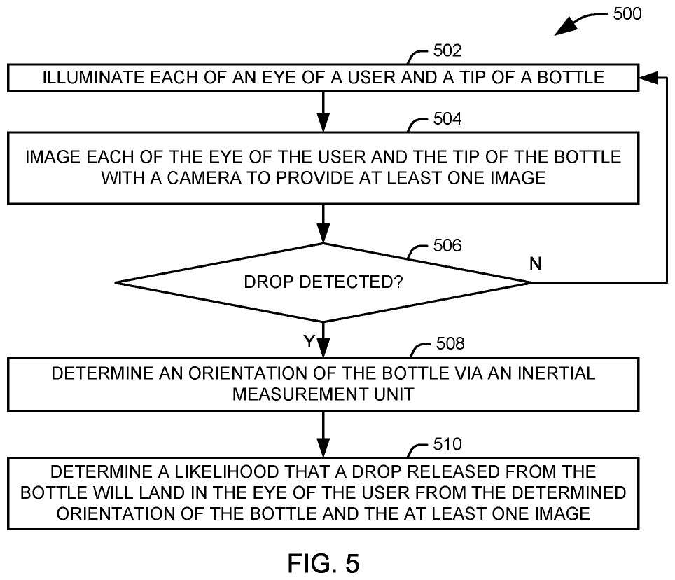

FIG. 5 illustrates a method 500 for monitoring compliance of user with a treatment protocol involving administration of drops to the eye of the user. At 502, each of an eye of a user and a tip of a bottle are illuminated. At 504, each of the eye of the user and the tip of the bottle are imaged with a camera to provide at least one image. In one example, each of eye of the user and the tip of the bottle are illuminated with light of a specific wavelength, and imaged through a spectral filter that attenuates light outside of a band of wavelengths including the specific wavelength. In another example, an illumination source is pulsed in synchrony with a frame acquisition rate of the camera to produce a plurality of illuminated images and a plurality of non-illuminated images, with an unilluminated image from each illuminated image to provide a background subtracted image during image processing. In a further example, the eye of the user is illuminated with a first illumination source and the tip of the bottle is illuminated with a second illumination source, with the first illumination source being inactive at least a portion of a time for which the second illumination source is active. For example, the tip of the bottle can be illuminated and imaged while the eye is not illuminated to reduce the effects of background illumination on images of the tip of the bottle.

At 506, it is determined from the at least one image if a drop has been released from the bottle. For example, a set of image processing techniques can be applied to the image to locate a reflection of an illumination source in the droplet. Alternatively, the images can be evaluated on a machine learning model trained on previous images or features extracted from images labeled with known outcomes. If no drop is detected (N), the method 500 returns to 502 to capture another image of the tip of the bottle and the eye. If a drop is detected (Y), the method 500 advances to 508, where an orientation of the bottle is determined via an inertial measurement unit. At 510, a likelihood that a drop released from the bottle will land in the eye of the user is determined from the determined orientation of the bottle and the at least one image. In one implementation, the at least one image and the determined orientation are provided to a machine learning model trained on previous data labelled with known outcomes to provide the likelihood that the drop will land in the eye. In another implementation, a relative location of the eye of the user and the tip of the bottle from the at least one image, and that relative location and the orientation of the bottle are used to determine the likelihood. The determined likelihood and a time at which the drop was released can be provided to the user via an output device and/or stored in a local or remote memory to represent compliance with a course of care.

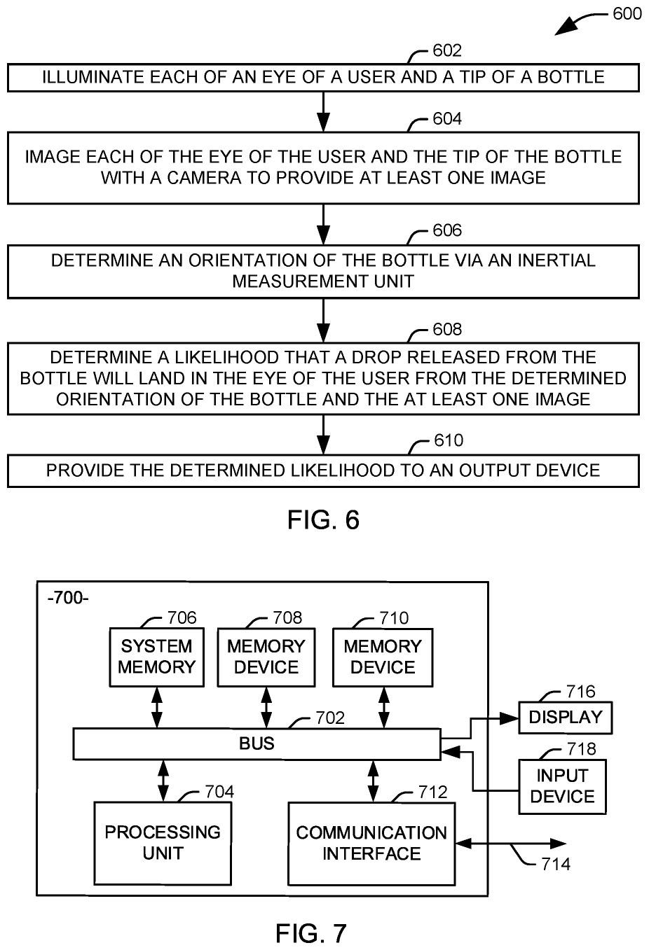

FIG. 6 illustrates a method 600 for determining if a released from a bottle with an eye dropper is likely to land in an eye of a user. At 602, each of an eye of a user and a tip of a bottle are illuminated. At 604, each of the eye of the user and the tip of the bottle are imaged with a camera to provide at least one image. In one example, each of eye of the user and the tip of the bottle are illuminated with light of a specific wavelength, and imaged through a spectral filter that attenuates light outside of a band of wavelengths including the specific wavelength. In another example, an illumination source is pulsed in synchrony with a frame acquisition rate of the camera to produce a plurality of illuminated images and a plurality of non-illuminated images, with an unilluminated image from each illuminated image to provide a background subtracted image during image processing. In a further example, the eye of the user is illuminated with a first illumination source and the tip of the bottle is illuminated with a second illumination source, with the first illumination source being inactive at least a portion of a time for which the second illumination source is active. For example, the tip of the bottle can be illuminated and imaged while the eye is not illuminated to reduce the effects of background illumination on images of the tip of the bottle.

At 606, an orientation of the bottle is determined via an inertial measurement unit. At 608, a likelihood that a drop released from the bottle will land in the eye of the user is determined from the determined orientation of the bottle and the at least one image. In one implementation, the at least one image and the determined orientation are provided to a machine learning model trained on previous data labelled with known outcomes to provide the likelihood that the drop will land in the eye. In another implementation, a relative location of the eye of the user and the tip of the bottle from the at least one image, and that relative location and the orientation of the bottle are used to determine the likelihood. At 610, the likelihood of the drop landing in the eye is provided to an output device. In one example, the output device provides one of an audible, visible, or tactile signal to the user representing the likelihood that the drop released from the bottle will land in the eye of the user. For example, a light emitting diode on the device can vary in color based on the determined likelihood to alert the user when a drop can be released with a high probability of successfully landing in the eye. In another example, the output device can be an automated mechanism that triggers a release of a drop from the bottle when the likelihood that the drop released from the bottle will land in the eye of the user exceeds a threshold value.

FIG. 7 is a schematic block diagram illustrating an example system 700 of hardware components capable of implementing examples of the systems and methods disclosed herein. For example, the system 700 can be used to implement the droplet detection component 208 and/or the alignment component 210 of FIG. 2 or the alignment component 108 of FIG. 1. The system 700 can include various systems and subsystems. The system 700 can include one or more of a personal computer, a laptop computer, a mobile computing device, a workstation, a computer system, an appliance, an application-specific integrated circuit (ASIC), a server, a server BladeCenter, a server farm, etc.

The system 700 can include a system bus 702, a processing unit 704, a system memory 706, memory devices 708 and 710, a communication interface 712 (e.g., a network interface), a communication link 2214, a display 716 (e.g., a video screen), and an input device 718 (e.g., a keyboard, touch screen, and/or a mouse). The system bus 702 can be in communication with the processing unit 704 and the system memory 706. The additional memory devices 708 and 710, such as a hard disk drive, server, standalone database, or other non-volatile memory, can also be in communication with the system bus 702. The system bus 702 interconnects the processing unit 704, the memory devices 706 and 710, the communication interface 712, the display 716, and the input device 718. In some examples, the system bus 702 also interconnects an additional port (not shown), such as a universal serial bus (USB) port.

The processing unit 704 can be a computing device and can include an application-specific integrated circuit (ASIC). The processing unit 704 executes a set of instructions to implement the operations of examples disclosed herein. The processing unit can include a processing core.

The additional memory devices 706, 708, and 710 can store data, programs, instructions, database queries in text or compiled form, and any other information that may be needed to operate a computer. The memories 706, 708 and 710 can be implemented as computer-readable media (integrated or removable), such as a memory card, disk drive, compact disk (CD), or server accessible over a network. In certain examples, the memories 706, 708 and 710 can comprise text, images, video, and/or audio, portions of which can be available in formats comprehensible to human beings.

Additionally, or alternatively, the system 700 can access an external data source or query source through the communication interface 712, which can communicate with the system bus 702 and the communication link 714.

In operation, the system 700 can be used to implement one or more parts of a system in accordance with the present invention, such as system 100, system 200, and/or measurement device 220. Computer executable logic for implementing the diagnostic system resides on one or more of the system memory 706, and the memory devices 708 and 710 in accordance with certain examples. The processing unit 704 executes one or more computer executable instructions originating from the system memory 706 and the memory devices 708 and 710. The term “computer readable medium” as used herein refers to a medium that participates in providing instructions to the processing unit 704 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

1. A method comprising:
illuminating each of an eye of a user and a tip of a bottle;
imaging each of the eye of the user and the tip of the bottle with a camera to provide at least one image;
determining an orientation of the bottle via an inertial measurement unit; and
determining a likelihood that a drop released from the bottle will land in the eye of the user from the determined orientation of the bottle and the at least one image.

2. The method of claim 1, further comprising detecting, from the at least one image, a release of a drop from the bottle, and determining the likelihood that the drop released from the bottle is performed in response to detecting the release of the drop from the bottle.

3. The method of claim 2, further comprising storing a time at which the drop was released and the determined likelihood that the drop released will land in the eye of the user at a non-transitory computer readable medium.

4. The method of claim 1, wherein illuminating each of eye of the user and the tip of the bottle comprises illuminating each of eye of the user and the tip of the bottle with light of a specific wavelength, and imaging each of the eye of the user and the tip of the bottle comprises imaging each of the eye of the user and the tip of the bottle through a spectral filter that attenuates light outside of a band of wavelengths including the specific wavelength.

5. The method of claim 1, wherein illuminating each of eye of the user and the tip of the bottle comprises modulating an illumination source to pulse in synchrony with a frame acquisition rate of the camera to produce a plurality of illuminated images and a plurality of non-illuminated images, and determining a likelihood that the drop released from the bottle will land in the eye of the user comprises subtracting an unilluminated image from each illuminated image to provide a background subtracted image.

6. The method of claim 1, wherein illuminating each of eye of the user and the tip of the bottle comprises illuminating the eye of the user with a first illumination source and illuminating the tip of the bottle with a second illumination source, the first illumination source being inactive at least a portion of a time for which the second illumination source is active.

7. The method of claim 1, wherein determining the likelihood that the drop released from the bottle will land in the eye of the user comprises providing the at least one image and the determined orientation to a machine learning model.

8. The method of claim 1, wherein determining the likelihood that the drop released from the bottle will land in the eye of the user comprises determining a relative location of the eye of the user and the tip of the bottle from the at least one image.

9. The method of claim 1, further comprising providing one of an audible, visible, or tactile signal to the user representing the likelihood that the drop released from the bottle will land in the eye of the user.

10. The method of claim 1, further comprising trigging a release of a drop from the bottle via an automated mechanism when the likelihood that the drop released from the bottle will land in the eye of the user exceeds a threshold value.

11. A system comprising:
an illumination source positioned to illuminate each of an eye of a user and a tip of a bottle when the when the device and the bottle with which it is associated is in an appropriate position to deliver mediation to the eye;
a camera that images each of the eye of the user and the tip of the bottle while the illumination source is active to provide at least one image;
an inertial measurement unit that determines an orientation of the bottle; and
an alignment component that determines a likelihood that a drop released from the bottle will land in the eye of the user from the determined orientation of the bottle and the at least one image.

12. The system of claim 11, further comprising a housing configured to engage with the bottle, the housing containing at least a portion of the alignment component.

13. The system of claim 11, further comprising an output device that provides one of an audible, visible, or tactile signal to the user representing the likelihood that the drop released from the bottle will land in the eye of the user.

14. The system of claim 11, further comprising trigging a release of a drop from the bottle via an automated mechanism when the likelihood that the drop released from the bottle will land in the eye of the user exceeds a threshold value.

15. The system of claim 11, further comprising a droplet detection component that processes images received from the camera to determine if the drop has been released from the tip of the bottle.

16. A method for monitoring compliance in the application of eye drops for a user, the method comprising:
illuminating each of an eye of a user and a tip of a bottle;
imaging each of the eye of the user and the tip of the bottle with a camera to provide at least one image;
detecting, from the at least one image, a release of a drop from the bottle;
determining an orientation of the bottle via an inertial measurement unit; and
determining a likelihood that a drop released from the bottle will land in the eye of the user from the determined orientation of the bottle and the at least one image in response to detecting the release of the drop from the bottle.

17. The method of claim 16, wherein illuminating each of eye of the user and the tip of the bottle comprises illuminating each of eye of the user and the tip of the bottle with light of a specific wavelength, and imaging each of the eye of the user and the tip of the bottle comprises imaging each of the eye of the user and the tip of the bottle through a spectral filter that attenuates light outside of a band of wavelengths including the specific wavelength.

18. The method of claim 16, wherein illuminating each of eye of the user and the tip of the bottle comprises modulating an illumination source to pulse in synchrony with a frame acquisition rate of the camera to produce a plurality of illuminated images and a plurality of non-illuminated images, and determining a likelihood that the drop released from the bottle will land in the eye of the user comprises subtracting an unilluminated image from each illuminated image to provide a background subtracted image.

19. The method of claim 16, wherein illuminating each of eye of the user and the tip of the bottle comprises illuminating the eye of the user with a first illumination source and illuminating the tip of the bottle with a second illumination source, the first illumination source being inactive at least a portion of a time for which the second illumination source is active.

20. The method of claim 16, further comprising storing a time at which the drop was released and the determined likelihood that the drop released will land in the eye of the user at a non-transitory computer readable medium.
